(19) United States
(12) Patent Application Publication
(10) Pub. No.: US 2022/0184113 A1
(43) Pub. Date: Jun. 16, 2022

(54) RATIONALLY DESIGNED ANTIVIRAL COMPOUNDS THAT INHIBIT SARS-CoV-2 AND THEIR METHODS OF USE THEREOF

(71) Applicant: The Regents of the University of Colorado, a body corporate, Denver, CO (US)

(72) Inventors: Anushree Chatterjee, Boulder, CO (US); Thomas R. Aunins, Boulder, CO (US); Prashant Nagpal, Lafayette, CO (US)

(21) Appl. No.: 17/546,177

(22) Filed: Dec. 9, 2021

Related U.S. Application Data
(60) Provisional application No. 63/123,563, filed on Dec. 10, 2020.

(51) Int. Cl. A61K 31/7105; A61K 38/10; C12N 15/113; A61P 31/14

(52) U.S. Cl. CPC A61K 31/7105; A61P 31/14; C12N 15/1131; A61K 38/10

(57) ABSTRACT

The inventive technology generally relates to systems, methods, and compositions for the treatment of viral infections, as well as novel use of antisense technology to rationally design antiviral compositions that can be applied to clinical cases and human infections. In one preferred aspect, the inventive technology includes methods, and compositions to treat COVID-19 in humans through the targeted inhibition of viral genome expression.

### STATEMENT OF FEDERALLY SPONSORED RESEARCH

This invention was made with government support under grant number NNX16A069A awarded by NASA. The government has certain rights in the invention.

### TECHNICAL FIELD

The inventive technology includes novel systems, methods, and compositions for the treatment and prevention of infection by the novel SARS-CoV-2 coronavirus (COVID-19). In particular, the inventive technology includes rationally designed antisense oligonucleotides for the targeted inhibition of genome expression in the SARS-CoV-2 coronavirus.

### BACKGROUND

The coronavirus disease 2019 (COVID-19) pandemic, which to date has resulted in more than 50 million cases and 1.2 million deaths globally, has exposed the urgent need for a reliable antiviral pipeline that can be used to rapidly develop treatments for novel pathogens. In the course of the pandemic, no antiviral specific to the SARS-CoV-2 virus has been made available, and existing direct-acting antiviral (DAA) drugs have proven to be moderately effective at best. Even outside of the ongoing crisis, the recent emergence of highly virulent strains combined with increasingly long development times for new vaccines and a relatively small catalog of existing DAA drugs—which is largely focused on treatments for hepatitis and HIV infections—presents a dangerous treatment gap that may potentially be filled using rational drug discovery strategies.

One such strategy is the genome-based drug design afforded by antisense oligomers, which allow for the inhibition of protein translation through sequence-specific binding to viral mRNA. These technologies have found moderate success in arresting viral replication both in vitro and in animal models, though the structural diversity of viral genomes requires a reassessment of design principles across species. Of particular interest within the antisense field are the nuclease-resistant phosphorodiamidate morpholino oligomers (PMO) and peptide nucleic acids (PNA), each of which is uncharged and binds with high affinity to RNA. Within the Coronaviridae virus family, the strain SARS-CoV—whose genome shows high degrees of homology with the SARS-CoV-2 virus—has been shown to be susceptible in vitro to PNA and PMO treatment. These studies demonstrate the utility of steric hindrance of RNA polymerase progression—the standard mechanism of PNA and PMO inhibition in both bacterial and mammalian gene expression inhibition—through binding to features such as the transcriptional regulatory sequence, translation start site, and ribosomal frameshift motif. Additionally, they suggest the potential utility of disrupting the viral RNA secondary structure in the coronavirus pseudoknot region.

In the present invention demonstrates the rapid design, synthesis, and testing of antisense peptide PNA for the treatment of COVID-19 via the Facile Accelerated Specific Therapeutic (FAST) platform. The application of the FAST platform, (generally described by Chatterjee A., et al., in PCT/US2020/045638, the figures, specification and claims being specifically incorporated herein by reference) allows for the rapid assessment of the inhibitory effect of antisense oligonucleotides, such as antisense PNA or PMO, being generally referred to as FASTmers, targeted to various regions of the SARS-CoV-2 genome and indicate which of these regions may offer the best target for antiviral therapies.

### SUMMARY OF THE INVENTION

The inventive technology generally relates to systems, methods, and compositions for the treatment of viral infections, as well as novel use of antisense technology to rationally design antiviral compositions that can be applied to clinical cases and human infections. In one preferred aspect, the inventive technology includes methods, and compositions to treat COVID-19 in humans through the targeted inhibition of viral gene expression.

One aspect of the inventive technology includes the rational design and use of antisense therapeutics that are complementary to one or more target sequences in the viral genome, wherein such antisense therapeutics bind to the target sequences and prevent viral gene expression, for example through steric interference with polymerase progression along the nucleotide strand. In a preferred aspect, the inventive technology includes the rational design and use of antisense FASTmers configured to be complementary to target sequences in the ssRNA genome of SAR-CoV-2 and inhibit viral gene expression. In another preferred aspect, the inventive technology includes the rational design and use of antisense FASTmers, such as antisense peptide-nucleic acid (PNA) FASTmer therapeutics configured to be complementary to target sequences in the ssRNA genome of SAR-CoV-2 and inhibit viral gene expression.

Another aspect of the inventive technology includes the use of rationally designed antisense PNA therapeutics to treat SAR-CoV-2 infection in a subject. In another aspect, the inventive technology includes the co-administration of rationally designed antisense PNA therapeutics with other therapeutic compositions to treat SAR-CoV-2 infection in a subject. In another aspect of the inventive technology includes the prophylactic use of rationally designed antisense therapeutics, such as antisense FASTmers, to prevent SAR-CoV-2 infection in a subject at risk of infection. In still further aspects, the inventive technology includes the prophylactic co-administration of rationally designed antisense, such as antisense FASTmers, with other prophylactic therapeutic compositions, such as vaccines, to prevent SAR-CoV-2 infection in a subject at risk of infection.

Another aspect of the invention includes antisense FASTmers configured to be complementary to a target sequence in the SAR-CoV-2 genome. In one preferred aspect, antisense FASTmers of the invention may be directed to target sequences, or regions, in the SAR-CoV-2 genome including, but not limited to: the transcriptional regulatory sequence (TRS), the start codon region (AUG), the polyprotein 1AB frameshift motif (FS), and the pseudoknot structure (PKI).

Another aspect of the invention includes antisense FASTmers configured to be complementary to a target sequence in the SAR-CoV-2 genome. In one preferred aspect, antisense FASTmers of the invention may be directed to target sequences, in the SAR-CoV-2 genome including, but not limited to: a target sequence in the transcriptional regulatory sequence (TRS) (SEQ ID NO. 7), a target sequence in the start codon region (AUG) (SEQ ID NO. 8), a target sequence in the polyprotein 1AB frameshift motif (FS) (SEQ ID NO. 9), and a target sequence in the pseudoknot structure (PKI) (SEQ ID NOs. 10-12).

Another aspect of the invention include antisense FASTmers configured to be complementary to a target sequence in the SAR-CoV-2 genome, and wherein said antisense FASTmers comprise antisense PNAs selected from the group consisting of: α-TRS (SEQ ID NO. 1), α-AUG (SEQ ID NO. 2), α-FS (SEQ ID NO. 3), and α-PK1-3 (SEQ ID NOs. 4-6). Another aspect of the invention includes the isolated PNAs, and corresponding antisense nucleotide sequences, according to SEQ ID NOs. 1-6.

Another aspect of the invention includes pharmaceutical compositions containing one or more antisense FASTmers configured to inhibit gene expression in SAR-CoV-2. In one preferred aspect, the invention includes methods of treating COVID-19 in a subject in need thereof, comprising the step of administering a therapeutically effective amount of a pharmaceutical compositions containing one or more antisense FASTmers configured to inhibit gene expression in SAR-CoV-2. In a preferred aspect, the invention includes methods of treating COVID-19 in a subject in need thereof, comprising the step of administering a therapeutically effective amount of a pharmaceutical compositions containing one or more antisense FASTmers configured to inhibit gene expression in SAR-CoV-2, selected from the group consisting of: α-TRS (SEQ ID NO. 1), (α-AUG (SEQ ID NO. 2), α-FS (SEQ ID NO. 3), and α-PK1-3 (SEQ ID NOs. 4-6).

The inventive technology further relates to a composition comprising an antisense FASTmers, and preferably an antisense PNA FASTmer and the use of the composition for the preparation of a pharmaceutical composition, especially a therapeutic compound, e.g. for use in the prophylaxis or treatment of COVID-19 coronavirus infection. The inventive technology further describes methods of treatment or prevention of infections of COVID-19 coronavirus in subjects in need thereof using the composition comprising an antisense FASTmers, and preferably an antisense PNA FASTmer configured to inhibit gene expression in SAR-CoV-2, selected from the group consisting of: α-TRS (SEQ ID NO. 1), (α-AUG (SEQ ID NO. 2), α-FS (SEQ ID NO. 3), and α-PK1-3 (SEQ ID NOs. 4-6). Another aspect of the invention includes the identification of novel targets sequences in the SAR-CoV-2 genome for the treatment of COVID-19.

Additional aspects of the invention may be evidenced from the specification, claims and figures provided below.

### BRIEF DESCRIPTION OF DRAWINGS

FIG. 1: shows selected target sequences for the positive strand ssRNA SARS-CoV-2 genome. One PNA each was selected for the transcriptional regulatory sequence (α-TRS) and the translation start region (α-AUG), and four were selected for the frameshift/pseudoknot region (α-FS, α-PK1-3).

FIG. 2: shows representative data of PNA assays in Vero E6 cells for cytotoxicity and infected cell recovery. Both metrics were analyzed using CellTiterGlo to measure cell viability in PNA concentrations ranging from <0.1 μM to 10 μM. Cytotoxicity was assessed via comparison of cell viability of treated cultures compared to untreated cultures (with 0% indicating equivalent viability and 100% indicating zero viability of the treated cultures). Viability of infected cells was assessed via comparison of treated infected cultures with untreated infected cultures (with 0% indicating no difference in viability and 100% indicating full recovery of the treated cultures).

FIG. 3: flow diagram of the Facile Accelerated Specific Therapeutic (FAST) platform generates antivirals (called FASTmers) against emerging viruses (here SARS-CoV2) with an accelerated design, build, test cycles of less than a week. Schematic showing the FAST process and timeline.

### DETAILED DESCRIPTION OF INVENTION

Generally, the inventive technology includes the use of predictive homology to rationally design novel antiviral therapeutic compositions, and in particular novel antisense antiviral therapeutic compositions used for the treatment of COVID-19 infection. In one preferred embodiment, the invention includes the identification or one or more target sequences of the SARS-CoV-2 genome, that when introduced to a complementary an antiviral therapeutic, inhibit viral gene expression. Once a target sequence has been identified, the invention further includes systems and methods of rational designing one or more antagomirs complementary to the genomic target sequence, which bind to, and inhibit viral gene expression of the SARS-CoV-2 coronavirus. These antagomirs or antisense inhibitors are also referred to herein as “FASTmers,” or “antisense FASTmers” and may include one or more antisense-oligonucleotides complementary to the genomic target sequence of SARS-CoV-2.

In one preferred embodiment, the “FASTmer” of the invention may include an antisense peptide nucleic acid (PNA) molecule designed to inhibit SARS-CoV-2 viral gene expression, and as a result inhibit viral replication in a subject. As used herein, PNAs may be DNA analogs in which the phosphate backbone has been replaced by (2-aminoethyl) glycine carboyl units that are linked to the nucleotide bases by the glycine amino nitrogen and methylene carbonyl linkers. The backbone is thus composed of peptide bonds linking the nucleobases. Because the PNA backbone is composed of peptide linkages, the PNA is typically referred to as having an amino-terminal and a carboxy-terminal end. However, a PNA can be also referred to as having a 5′ and a 3′ end in the conventional sense, with reference to the complementary nucleic acid sequence to which it specifically hybridizes. The sequence of a PNA molecule is described in conventional fashion as having nucleotides G, U, T, A, and C that correspond to the nucleotide sequence of the DNA molecule. Such polynucleotides can be synthesized, for example, using an automated DNA synthesizer. Typically, PNAs are synthesized using either Boc or Fmoc chemistry. PNAs and other polynucleotides can be chemically derivatized by methods known to those skilled in the art. For example, PNAs have amino and carboxy groups at the 5′ and 3′ ends, respectively, that can be further derivatized. Custom PNAs can also be synthesized and purchased commercially. Since PNA is structurally markedly different from DNA, PNA is very resistant to both proteases and nucleases, and is not recognized by the hepatic transporter(s) recognizing DNA. However, it should be noted that in one preferred embodiment a FASTmer is a PNA oligomer directed to a target sequence of SARS-CoV-2, in alternative embodiments a FASTmer may include any antisense, or other oligomer, that may bind to a target sequence of SARS-CoV-2 and inhibit viral gene expression or replication.

One embodiment of the inventive technology includes the rational design and use of antisense therapeutics configured to be complementary to viral genomic target sequences, and in particular genomic target sequences of SARS-CoV-2. In another preferred embodiment, the inventive technology includes the rational design and use of antisense FASTmers, such as antisense peptide-nucleic acid (PNA) FASTmer therapeutics to be complementary to viral genomic target sequences, and in particular SARS-CoV-2 genomic regions involved in viral gene expression and replication.

In some embodiments, a PNA FASTmer for treating a COVID-19 coronavirus infection includes at least one, at least two, at least three, or at least four or more antisense PNAs FASTmers configured to inhibit viral gene expression in SARS-CoV-2, wherein each antisense PNA FASTmer includes a sequence of at least 5, and up to 20 or more nucleic acids capable of hybridizing to a target sequence. In some embodiments, the antisense PNA molecules of the PNA system may be configured to target a specific genomic region, motif or structure, which may be selected from the group consisting of: the transcriptional regulatory sequence (TRS), the start codon region (AUG), the polyprotein 1AB frameshift motif (α-FS), and the pseudoknot structure (PKI).

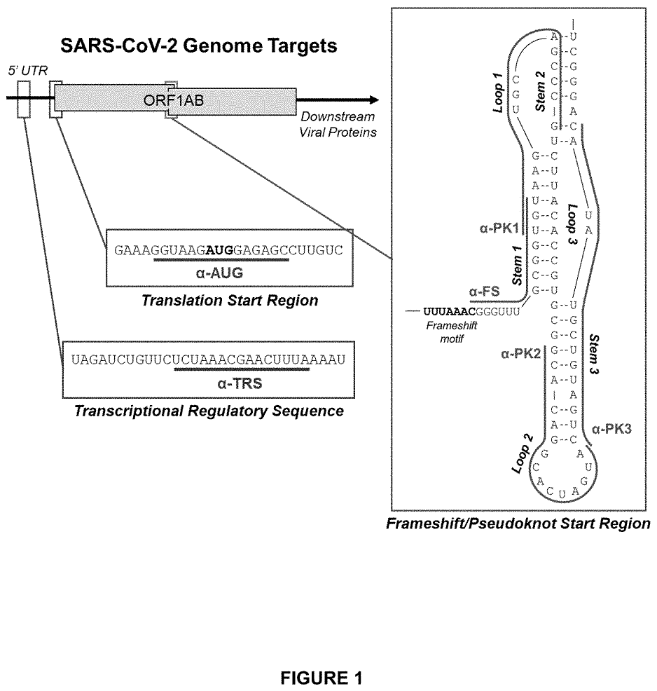

It will be recognized by those of skill in the art that any of the nucleotide sequences described above can be targeted by antisense inhibitors. Target sequences can a viral DNA or RNA sequence, such as the positive strand ssRNA genome of SARS-CoV-2. Given the benefit of this disclosure, those of skill in the art will be able to identify a target sequence and design an antisense inhibitor oligomer that is complementary to the nucleotide sequence. Inhibition can be caused by steric interference resulting from an antisense oligomer binding the target sequence, thereby preventing the targets function or activity, for example through the steric interference with transcriptional machinery in the host. Target sites, such as those identified in FIG. 1, can be any site to which binding of an antisense oligomer will inhibit transcription of the viral genome, and preferably transcription of one or more regions of the SARS-CoV-2 genome.

An antisense FASTmer oligomer can be complementary to a single target sequence or to two or more target sequences. Alternatively, as shown in Table 1, multiple antisense FASTmer oligomers, such as α-PKI (SEQ ID NOs. 1-6) can target a single target regions that may be part of a motif or structure, such as the 1AB frameshift motif (FS), and the pseudoknot structure (PKI). In particular embodiments, each individual antisense oligomer is complementary to a single target site. Wherein each individual antisense oligomer is complementary to a single target site, the antisense oligomer can be about less than 10-mers, 10-mers to 20-mers in length, or greater than 20-mers. In certain embodiments, the antisense FASTmer oligomer is 15-mers in length. In certain embodiments, an antisense FASTmer oligomer is designed with the target sequence in the middle of the oligomer, with 3-5 nucleotides flanking the overlapping region. This provides for antisense oligomers with both high affinity and specificity. Wherein an individual antisense oligomer is complementary to two or more target sites, the antisense oligomer can be up to about 40-mers or greater in length.

Another embodiment of the inventive technology includes the use of rationally designed antisense PNA therapeutics to treat SAR-CoV-2 infection in a subject. In another embodiment of the inventive technology includes the co-administration of rationally designed antisense PNA therapeutics with other therapeutic compositions to treat SAR-CoV-2 infection in a subject. In yet another embodiment, of the inventive technology includes the prophylactic use of rationally designed antisense PNA therapeutics to prevent SAR-CoV-2 infection in a subject at risk of infection. In still further embodiments, the inventive technology includes the prophylactic co-administration of rationally designed antisense PNA therapeutics with other prophylactic therapeutic compositions, such as vaccines, to prevent SAR-CoV-2 infection in a subject at risk of infection. Another embodiment of the invention includes antisense FASTmers configured to be complementary to a target sequence in the SAR-CoV-2 genome, such complementary binding causing the inhibition of viral gene expression in a host. Another embodiment of the invention include antisense FASTmers configured to be complementary to a target sequence in the SAR-CoV-2 genome, and wherein said antisense FASTmers comprise antisense PNAs according to SEQ ID NOs. 1-6, which are configured to bind to such target sequences and inhibit viral gene expression thereby preventing viral replication in the host.

A composition of the present disclosure can comprise one or more antisense FASTmers oligomers, such as a rationally designed antisense PNA configured to inhibit viral genome transcription of SAR-CoV-2 and may further have antiviral therapeutic effects. For example, a composition can comprise an antisense PNA FASTmers according to SEQ ID NOs 1-6, directed to one or more viral genomic target sequences, structures or motifs, and in particular: the transcriptional regulatory sequence (TRS), the start codon region (AUG), the polyprotein 1AB frameshift motif (α-FS), and the pseudoknot structure (PKI).

Another embodiment of the invention includes the isolated FASTmer sequences according to SEQ ID NOs. 1-6, and preferably where said FASTmer sequences according to SEQ ID NOs. 1-6 comprise PNA or PMO sequences. Another embodiment of the invention includes methods of producing the isolated FASTmer sequences according to SEQ ID NOs. 1-6, and preferably where said FASTmer sequences according to SEQ ID NOs. 1-6 comprise PNA or PMO sequences.

As noted, such compositions can also have antiviral therapeutic effects, and may be particularly effective at treating infection caused by SAR-CoV-2. At least one antisense FASTmers may be present in the composition at a therapeutically effective amount. The therapeutically effective amount of an antisense FASTmers will depend on several factors, including but not limited to the FASTmers's backbone composition, the affinity of the FASTmers for its target, the specificity of the FASTmers for its target, and the ability of the FASTmers to enter the cell. In certain embodiments, a therapeutically effective amount of an antisense FASTmers is that concentration that prevents or treats COVID-19 infection in a subject in need thereof.

In certain embodiments, a FASTmer composition of the invention is a pharmaceutical composition. The compositions may routinely contain pharmaceutically acceptable concentrations of salt, buffering agents, preservatives and various compatible carriers or diluents. The proportion and identity of the pharmaceutically acceptable diluent is determined by chosen route of administration, compatibility with the antisense FASTmer oligomers of the composition, and standard pharmaceutical practice. Generally, the pharmaceutical composition will be formulated with components that will not significantly impair the biological activities of the antisense FASTmer oligomers. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the compositions are prepared uniformly and intimately, bringing into association the active ingredients with liquid carriers or finely divided solid carriers or both, and then, if necessary, shaping the product. One of skill in the art will recognize that formulations are routinely designed according to their intended use, i.e. route of administration.

Wherein the composition comprises one or more antisense FASTmer oligomers having antiviral, and in particular anti-COVID-19 effects, a subject can be treated for bacterial infection by administering an appropriate dose of the composition. Compositions described herein can be administered similarly to currently available antivirals or antibiotics, including but not limited to oral administration, nasal administration, intravenous administration, intramuscular administration, intraperitoneal administration, topical administration, local delivery methods, and in feed and water supplies. The formulation of therapeutic compositions and their subsequent administration (dosing) is believed to be within the skill of those in the art.

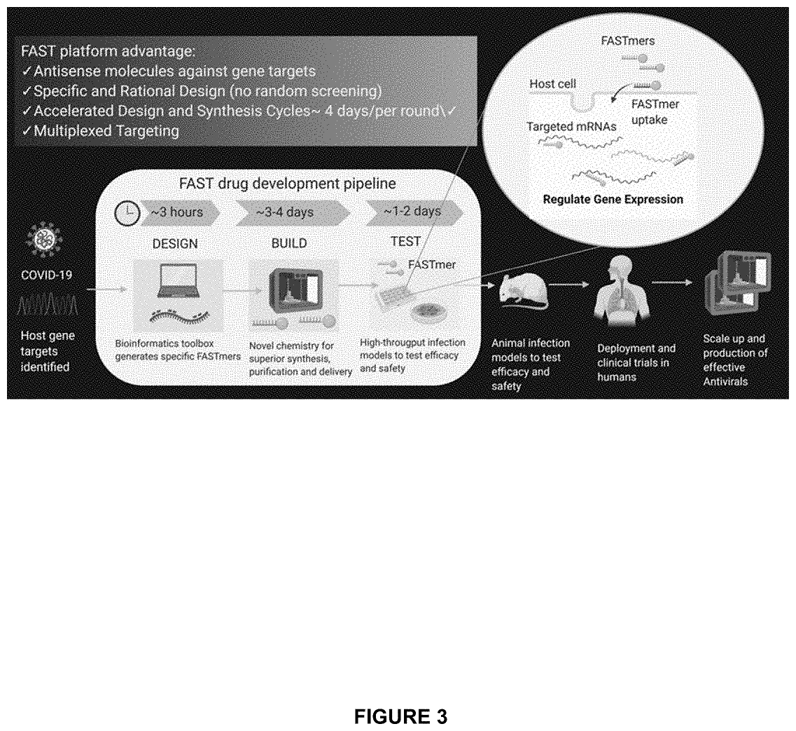

Optimal dosing schedules can be calculated from measurements of drug accumulation in the body of a subject. Persons of ordinary skill can easily determine optimum dosages, dosing methodologies and repetition rates. Optimum dosages may vary depending on the relative potency of individual PNA FASTmers and can generally be estimated based on EC₅₀s found to be effective in in vitro and in vivo animal models as shown in FIG. 3. In general, dosage is from 0.01 μg to 100 g per kg of body weight, and may be given once or more daily, weekly, or monthly. Persons of ordinary skill in the art can easily estimate repetition rates for dosing based on measured residence times and concentrations of the antisense oligomers in bodily fluids or tissues. Following successful treatment, it may be desirable to have the subject undergo maintenance therapy to prevent the recurrence of the disease state, wherein one or more PNAs is administered in maintenance doses, ranging from 0.01 μg to 100 g per kg of body weight. In certain embodiments, a patient is treated with a dosage of one or more PNAs that is at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100 mg/kg body weight.

Another embodiment of the invention includes pharmaceutical compositions containing one or more antisense FASTmers configured to inhibit transcription of the SAR-CoV-2 genome. In one preferred embodiment, the invention includes methods of treating COVID-19 in a subject in need thereof, comprising the step of administering a therapeutically effective amount of a pharmaceutical composition containing one or more antisense FASTmers configured to inhibit transcription of the SAR-CoV-2 genome. As described above, the antisense FASTmers can be used in a synergistic combination with other known antiviral agents, and in particular anti-COVID-19 agents such as Remdesivir®, convalescent plasma, or one or more developmental vaccines directed towards SARS-CoV-2.

The inventive technology further comprises an antisense FASTmers, and preferably an antisense PNA FASTmer and the use of the composition for the preparation of a pharmaceutical composition, especially a therapeutic compound, e.g. for use in the prophylaxis or treatment of COVID-19 coronavirus infection. The inventive technology further describes methods of treatment or prevention of infections of COVID-19 coronavirus in subjects in need thereof using the composition comprising an antisense FASTmers, and preferably an antisense PNA FASTmer. Subjects to be treated for a COVID-19 infection can be selected from the group of: human; feed animals including but not limited to cattle, swine, poultry, goat, and sheep; companion animals, and laboratory animals.

Another embodiment of the invention includes pharmaceutical compositions containing one or more antisense PNA FASTmers configured to inhibit transcription of the SAR-CoV-2 genome. In one preferred embodiment, the invention includes methods of treating COVID-19 in a subject in need thereof, comprising the step of administering a therapeutically effective amount of a pharmaceutical compositions containing one or more antisense PNA FASTmers, and preferably one or more antisense PNA FASTmers selected from the group consisting of SEQ ID NO NOs. 1-6, wherein the antisense PNA FASTmers are configured to inhibit transcription of the SAR-CoV-2 genome. Another embodiment of the invention includes the identification of novel genomic targets for the treatment of viral infection, and in particular novel genomic target sequences for the treatment of COVID-19, such as target sequences selected from the group consisting of: TRS, AUG, FS, and PK1-3.

In one embodiment, at least one FASTmer, and preferably an antisense FASTmer configured to inhibit genomic expression in SARS-CoV-2 may be delivered to a subject in need thereof through a lipid nanoparticles. Preferably, lipid nanoparticles (LNPs) comprise: (a) at least one FASTmer, and preferably an antisense FASTmer configured to inhibit genomic expression in SARS-CoV-2, optionally comprised by the (pharmaceutical) composition as defined herein, (b) a cationic lipid, (c) optionally an aggregation reducing agent (such as polyethylene glycol (PEG) lipid or PEG-modified lipid), (d) optionally a non-cationic lipid (such as a neutral lipid), and (e) optionally, a sterol. In the context of the present invention, the term “lipid nanoparticle”, also referred to as “LNP”, is not restricted to any particular morphology, and includes any morphology generated when a cationic lipid and optionally one or more further lipids are combined, e.g. in an aqueous environment and/or in the presence of an antisense FASTmer. For example, a liposome, a lipid complex, a lipoplex, an emulsion, a micelle, a lipidic nanocapsule, a nanosuspension and the like are within the scope of a lipid nanoparticle (LNP). In some embodiments, LNPs comprise, in addition to the at least one FASTmer, and preferably an antisense FASTmer configured to inhibit genomic expression in SARS-CoV-2, optionally comprised by the (pharmaceutical) composition as defined herein, (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

In some embodiments, the inventive FASTmer, and preferably an antisense FASTmer configured to inhibit genomic expression in SARS-CoV-2, optionally comprised by the (pharmaceutical) composition, may be formulated in an aminoalcohol lipidoid. Aminoalcohol lipidoids which may be used in the present invention may be prepared by the methods described in U.S. Pat. No. 8,450,298, herein incorporated by reference in its entirety. LNPs may include any cationic lipid suitable for forming a lipid nanoparticle. Preferably, the cationic lipid carries a net positive charge at about physiological pH. The cationic lipid may be an amino lipid. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Ci), 1,2-Dilinoleoyi-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3 aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-DMA), (6Z,9Z,28Z,31Z)-heptatriaconta-6,9,28,31-tetraen-19-yl 4-(dim ethyl amino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P-(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DO SPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other suitable (cationic) lipids are disclosed in WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601. In that context, the disclosures of WO2009/086558, WO2009/127060, WO2010/048536, WO2010/054406, WO2010/088537, WO2010/129709, WO2011/153493, US2011/0256175, US2012/0128760, US2012/0027803, and U.S. Pat. No. 8,158,601 are incorporated herewith by reference. In some aspects the lipid may be selected from the group consisting of 98N12-5, C12-200, and ckk-E12.

The cationic lipid may also be an amino lipid. Suitable amino lipids include those having alternative fatty acid groups and other dialkylamino groups, including those in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid. Representative amino lipids include, but are not limited to, 1,2-dilinoleyoxy-3-(dimethylamino)acetoxypropane (DLin-DAC), 1,2-dilinoleyoxy-3 morpholinopropane (DLin-MA), 1,2-dilinoleoyl-3-dimethylaminopropane (DLinDAP), 1,2-dilinoleylthio-3-dimethylaminopropane (DLin-S-D A), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); C3 (US20100324120).

In some embodiments, amino or cationic lipids have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention. In some embodiments, the protonatable lipids have a pKa of the protonatable group in the range of about 4 to about 11, e.g., a pKa of about 5 to about 7. LNPs can include two or more cationic lipids. The cationic lipids can be selected to contribute different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid is from about 3 to about 15, such as from about 5 to about 13 or from about 7 to about 11. The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the antisense FASTmer which is used as cargo. The N/P ratio may be calculated on the basis that, for example, Ipg RNA typically contains about 3 nmol phosphate residues, provided that the antisense FASTmer exhibits a statistical distribution of bases. The “N”-value of the lipid or lipidoid may be calculated on the basis of its molecular weight and the relative content of permanently cationic and—if present—cationisable groups. In certain embodiments, the LNP comprises one or more additional lipids which stabilize the formation of particles during their formation.

In some embodiments, non-cationic may be used. The non-cationic lipid can be a neutral lipid, an anionic lipid, or an amphipathic lipid. Neutral lipids, when present, can be any of a number of lipid species which exist either in an uncharged or neutral zwitterionic form at physiological pH. Such lipids include, for example, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., LNP size and stability of the LNP in the bloodstream. Preferably, the neutral lipid is a lipid having two acyl groups (e.g., diacylphosphatidylcholine and diacylphosphatidylethanolamine). In some embodiments, the neutral lipids contain saturated fatty acids with carbon chain lengths in the range of C₁₀ to C₂₀. In other embodiments, neutral lipids with mono or diunsaturated fatty acids with carbon chain lengths in the range of CIO to C20 are used. Additionally, neutral lipids having mixtures of saturated and unsaturated fatty acid chains can be used. Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-0-monomethyl PE, 16-O-dimethyl PE, 18-1-trans PE, 1-stearoyl-2-oleoyl-phosphatidyethanolamine (SOPE), cholesterol, or a mixture thereof. Anionic lipids suitable for use in LNPs include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids. In one embodiment, the neutral lipid is 1,2-distearoyl-sn-glycero-3phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1. Amphipathic lipids refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, paimitoyloleoyl phosphatdylcholine, A phosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and beta-acyloxyacids, can also be used.

In some embodiments, the non-cationic lipid is present in a ratio of from about 5 mol % to about 90 mol %, about 5 mol % to about 10 mol %, about 5, 10, 15, 20, 25, 30, 35, 40, 45, 50, 55, 60, 65, 70, 75, 80, 85, or about 90 mol % of the total lipid present in the LNP. In some embodiments, LNPs comprise from about 0% to about 15 or 45% on a molar basis of neutral lipid, e.g., from about 3 to about 12% or from about 5 to about 10%. For instance, LNPs may include about 15%, about 10%, about 7.5%, or about 7.1% of neutral lipid on a molar basis (based upon 100% total moles of lipid in the LNP).

In some embodiments, a sterol may be used. The sterol is preferably cholesterol. The sterol can be present in a ratio of about 10 mol % to about 60 mol % or about 25 mol % to about 40 mol % of the LNP. In some embodiments, the sterol is present in a ratio of about 10, 15, 20, 25, 30, 35, 40, 45, 50, 55, or about 60 mol % of the total lipid present in the LNP. In other embodiments, LNPs comprise from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the LNP). In some embodiments, an aggregation reducing agent may be employed. The aggregation reducing agent can be a lipid capable of reducing aggregation. Examples of such lipids include, but are not limited to, polyethylene glycol (PEG)-modified lipids, monosialoganglioside Gml, and polyamide oligomers (PAO) such as those described in U.S. Pat. No. 6,320,017, which is incorporated by reference in its entirety. Other compounds with uncharged, hydrophilic, steric-barrier moieties, which prevent aggregation during formulation, like PEG, Gml or ATTA, can also be coupled to lipids. ATTA-lipids are described, e.g., in U.S. Pat. No. 6,320,017, and PEG-lipid conjugates are described, e.g., in U.S. Pat. Nos. 5,820,873, 5,534,499, 5,885,613, US20150376115A1 and WO2015/199952, each of which is incorporated by reference in its entirety.

The aggregation reducing agent may be, for example, selected from a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof (such as PEG-Cer14 or PEG-Cer20). The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids include, but are not limited to, polyethylene glycol-didimyristoyl glycerol (C14-PEG or PEG-C14, where PEG has an average molecular weight of 2000 Da) (PEG-DMG); (R)-2,3-bis(octadecyloxy)propyl-1-(methoxy polyethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl-1-(methoxy polyethylene glycol)2000)propylcarbamate)) (GalNAc-PEG-DSG); mPEG (mw2000)-diastearoylphosphatidyl-ethanolamine (PEG-DSPE); and polyethylene glycol-dipalmitoylglycerol (PEG-DPG). In some embodiments, the aggregation reducing agent is PEG-DMG. In other embodiments, the aggregation reducing agent is PEG-c-DMA.

In various embodiments, the molar ratio of the cationic lipid to the PEGylated lipid ranges from about 100:1 to about 25:1. In a preferred embodiment, the composition of LNPs may be influenced by, inter alia, the selection of the cationic lipid component, the degree of cationic lipid saturation, the nature of the PEGylation, the ratio of all components and biophysical parameters such as its size. In one example by Semple et al. (Semple et al. Nature Biotech. 201028: 172-176; herein incorporated by reference in its entirety), the LNP composition was composed of 57.1% cationic lipid, 7.1% dipalmitoylphosphatidylcholine, 34.3% cholesterol, and 1.4% PEG-c-DMA (Basha et al. Mol Ther. 2011 19:2186-2200; herein incorporated by reference in its entirety). In some embodiments, LNPs may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to FASTmer may range from about 5:1 to about 20:1, from about 10:1 to about 25:1, from about 15:1 to about 30:1 and/or at least 30:1. The average molecular weight of the PEG moiety in the PEG-modified lipids can range from about 500 to about 8,000 Daltons (e.g., from about 1,000 to about 4,000 Daltons). In one preferred embodiment, the average molecular weight of the PEG moiety is about 2,000 Daltons.

The concentration of the aggregation reducing agent may range from about 0.1 to about 15 mol %, per 100% total moles of lipid in the LNP. In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). Different LNPs having varied molar ratios of cationic lipid, non-cationic (or neutral) lipid, sterol (e.g., cholesterol), and aggregation reducing agent (such as a PEG-modified lipid) on a molar basis (based upon the total moles of lipid in the lipid nanoparticles).

The total amount of nucleic acid, particularly the one or more antisense FASTmers in the lipid nanoparticles varies and may be defined depending on the e.g. an antisense FASTmer to total lipid w/w ratio. In one embodiment of the invention the antisense FASTmer to total lipid ratio is less than 0.06 w/w, preferably between 0.03 w/w and 0.04 w/w.

In some embodiments, LNPs occur as liposomes or lipoplexes as described in further detail below. In some embodiments, LNPs have a median diameter size of from about 50 nm to about 300 nm, such as from about 50 nm to about 250 nm, for example, from about 50 nm to about 200 nm. In some embodiments, smaller LNPs may be used. Such particles may comprise a diameter from below 0.1 pm up to 100 nm such as, but not limited to, less than 0.1 pm, less than 1.0 pm, less than 5 pm, less than 10 pm, less than 15 pm, less than 20 pm, less than 25 pm, less than 30 pm, less than 35 pm, less than 40 pm, less than 50 pm, less than 55 pm, less than 60 pm, less than 65 pm, less than 70 pm, less than 75 pm, less than 80 pm, less than 85 pm, less than 90 pm, less than 95 pm, less than 100 pm, less than 125 pm, less than 150 pm, less than 175 pm, less than 200 pm, less than 225 pm, less than 250 pm, less than 275 pm, less than 300 pm, less than 325 pm, less than 350 pm, less than 375 pm, less than 400 pm, less than 425 pm, less than 450 pm, less than 475 pm, less than 500 pm, less than 525 pm, less than 550 pm, less than 575 pm, less than 600 pm, less than 625 pm, less than 650 pm, less than 675 pm, less than 700 pm, less than 725 pm, less than 750 pm, less than 775 pm, less than 800 pm, less than 825 pm, less than 850 pm, less than 875 pm, less than 900 pm, less than 925 pm, less than 950 pm, less than 975 pm, In another embodiment, nucleic acids may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nm, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. In some embodiments, the LNP may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In other embodiments, LNPs have a single mode particle size distribution (i.e., they are not bi- or poly-modal). LNPs, as used herein may further comprise one or more lipids and/or other components in addition to those mentioned above.

Other lipids may be included in the liposome compositions for a variety of purposes, such as to prevent lipid oxidation or to attach ligands onto the liposome surface. Any of a number of lipids may be present in LNPs, including amphipathic, neutral, cationic, and anionic lipids. Such lipids can be used alone or in combination. Additional components that may be present in an LNP include bilayer stabilizing components such as polyamide oligomers (see, e.g., U.S. Pat. No. 6,320,017, which is incorporated by reference in its entirety), peptides, proteins, and detergents.

In some embodiments, the inventive FASTmers, optionally comprised by (pharmaceutical) compositions are formulated as liposomes. Cationic lipid-based liposomes are able to complex with negatively charged nucleic acids (e.g. FASTmers) via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the nucleic acid is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Liposomes typically consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposomes may have one or more lipid membranes. Liposomes can be single-layered, referred to as unilamellar, or multi-layered, referred to as multilamellar. Liposome characteristics and behavior in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286). Liposomes are typically present as spherical vesicles and can range in size from 20 nm to a few microns. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety. The inventive FASTmer, optionally comprised by the (pharmaceutical) composition, may be encapsulated by the liposome and/or it may be contained in an aqueous core which may then be encapsulated by the liposome (see International Pub. Nos. WO2012/031046, WO2012/031043, WO2012/030901 and WO2012/006378 and US Patent Publication No. US20130189351, US20130195969 and US20130202684; the contents of each of which are herein incorporated by reference in their entirety).

In some embodiments, the inventive FASTmer, and preferably an antisense FASTmer configured to inhibit genomic expression in SARS-CoV-2, optionally comprised by the (pharmaceutical) composition, may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for (Landen et al. Cancer Biology & Therapy 2006 5(12)1708-1713); herein incorporated by reference in its entirety) and hyaluronan-coated liposomes (Quiet Therapeutics, Israel).

In some embodiments, the inventive FASTmer, and preferably an antisense FASTmer configured to inhibit genomic expression in SARS-CoV-2, optionally comprised by the (pharmaceutical) composition, is formulated in the form of lipoplexes, i.e. cationic lipid bilayers sandwiched between nucleic acid (e.g. FASTmer) layers. Cationic lipids, such as DOTAP, (1,2-dioleoyl-3-trimethylammonium-propane) and DOTMA (N-[1-(2,3-dioleoyloxy)propyl]-N,N,N-trimethylammonium methyl sulfate) can form complexes or lipoplexes with negatively charged nucleic acids to form nanoparticles by electrostatic interaction, providing high in vitro transfection efficiency.

In some embodiments, the inventive FASTmer, and preferably an antisense FASTmer configured to inhibit genomic expression in SARS-CoV-2, optionally comprised by the (pharmaceutical) composition as defined herein, is formulated in the form of nanoliposomes, preferably neutral lipid-based nanoliposomes such as 1,2-dioleoyl-sn-glycero-3-phosphatidylcholine (DOPC)-based nanoliposomes (Adv Drug Deliv Rev. 2014 February; 66: 110-116). In some embodiments, the inventive FASTmer, and preferably an antisense FASTmer configured to inhibit genomic expression in SARS-CoV-2, optionally comprised by the (pharmaceutical) composition as defined herein, is provided in the form of an emulsion. In some embodiment, said FASTmer is formulated in a cationic oil-in-water emulsion, wherein the emulsion particle comprises an oil core and a cationic lipid which can interact with said FASTmer, anchoring the molecule to the emulsion particle (see International Pub. No. WO2012/006380; herein incorporated by reference in its entirety). In some embodiments, said FASTmer is formulated in a water-in-oil emulsion comprising a continuous hydrophobic phase in which the hydrophilic phase is dispersed. As a non-limiting example, the emulsion may be made by the methods described in International Publication No. WO2010/87791, the contents of which are herein incorporated by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Antisense FASTmers Improve Mammalian Cell Survival for In Vitro SARS-CoV-2 Infections

The present inventors, utilizing the FAST platform, rationally designed, built, and tested a plurality of antisense FASTmer design within less than a single week. First, the PNA Finder toolbox was used to find potential antisense targets in the following regions of the SARS-CoV-2 viral genome (Genbank: MN908947.3): the transcriptional regulatory sequence (TRS), the start codon region, the polyprotein 1AB frameshift motif, and the pseudoknot structure. Multiple 15-mer PNA candidates were derived from each region of the genome, and were filtered for high predicted aqueous solubility, according to solubility metrics previously established in the art, as well as a lack of self-complementing sequences. Each sequence was also screened for potential incidental alignments within the viral genome (allowing up to two mismatches), and those that were found to have alternate potential alignments were eliminated.

The PNA candidates that fulfilled each of these criteria were then analyzed using PNA Finder for potential inhibitory off-targets (those predicted to suppress a given gene's expression) within both the human genome (GRCh38.p12, Genbank: GCA_000001405.27) and the green monkey genome (Chlorocebus sabeus 1.1, Genbank: GCA_000409795.2), as the green monkey-derived Vero E6 cell line were used as an in vitro infection model. These analyses were used to select PNA sequences that would minimize the potential for inhibitory off-target alignments in both species. Additionally, target sequence candidates were evaluated to maximize target sequence homology with 178 viral clinical isolates collected by the National Center for Biotechnology Information (accessed on Mar. 30, 2020). A nonsense sequence PNA oligomer was designed using the same criteria (excluding the clinical isolate homology) to be used as a negative control. In total, the compilation of viral target sequences, analysis of solubility and self-complement, and the search for off-target alignments took one day to complete.

As generally shown in FIG. 1, six antisense PNA FASTmers were selected: one targeting the TRS (α-TRS) (SEQ ID NO. 1), one targeting the start codon (α-AUG) (SEQ ID NO. 2), one targeting the 1AB frameshift motif (α-FS) (SEQ ID NO. 3), and three targeting the pseudoknot (α-PK1-3) (SEQ ID NOs. 4-6) (See also Table 1). These latter three were targeted to the pseudoknot region's stem 1/loop 1/stem 2, loop 2/stem 3, and stem 3/loop 3, respectively, in order to assess the viability of targeting different secondary structure features within this region. Of the six selected PNA, only one (α-AUG) was predicted to have an inhibitory off-target within the human genome (a gene coding for collagen type XXV alpha 1 chain), and the presence of a mismatched base-pair within this off-target would be expected to substantially abrogate binding (See Table 1). Additionally, in analysis of clinical isolate homology, only α-TRS and α-AUG target sequences with a mutation present in the strains examined, and each sequence is mutated at only a single base position in only one strain.

The PNA were synthesized using Fmoc chemistry and purified and further conjugated with nanoparticles, as these have been demonstrated in previous work to improve transport in mammalian systems. The process of synthesis, conjugation, and purification was completed in less than 4 days.

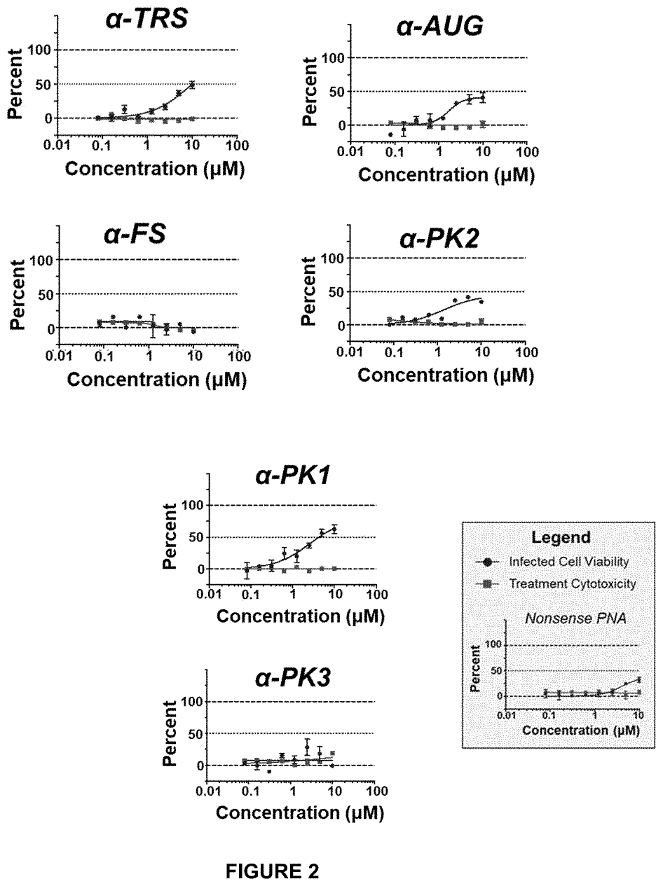

As shown in FIG. 2, the antisense PNA FASTmers (SEQ ID NOs. 1-6) were tested for both cytotoxicity and their ability to restore viability (assayed using CellTiterGlo) to mammalian cells infected with SARS-CoV-2. Cytotoxicity was measured by incubating non-infected Vero E6 cell cultures with varying antisense PNA FASTmer concentrations up to 10 μM and comparing the measured viability to cultures without treatment after three days of treatment. No cytotoxic effect was identified at any concentration. Viability restoration was studied by incubating Vero E6 cell cultures with each antisense PNA FASTmer at concentrations varying from 0.1 μM to 10 μM for two hours prior to inoculation with the SARS-CoV-2 virus at an MOI of 0.01. As with the cytotoxicity assay, these infections were allowed to proceed for three days before cell viability was measured and compared to infected controls that were not treated with PNA.

Again, as demonstrated in FIG. 2, multiple antiviral PNA FASTmers were found to produce a dose-dependent response in cell viability, used here as a cell survival metric to infer efficacy of viral replication inhibition. Of these, the α-PK1 (SEQ ID NO. 4) antisense FASTmer induced the greatest recovery in cell viability (IC₅₀=4.5 μM), and both α-TRS (SEQ ID NO. 1) and α-AUG (SEQ ID NO. 2) also produced close to 50% recovery in cell viability at the concentration 10 μM. The PNAs α-FS (SEQ ID NO. 3) and α-PK2 (SEQ ID NO. 5) demonstrated lower levels of recovery of cell viability at this concentration, and none was observed for α-PK3 (SEQ ID NO. 6). These data, together with the lack of viability improvement in the nonsense PNA treatment, suggest a sequence specificity of these antisense treatments in exemplary mammalian Vero E6 cells.

### Definitions

For the sake of clarity and readability, the following scientific background information and definitions are provided. Any technical features disclosed thereby can be part of each and every embodiment of the invention. Additional definitions and explanations can be provided in the context of this disclosure.

As used herein, “antisense oligomers” or “antisense oligonucleotide” means any antisense molecule that may modulate the expression of one or more genes. Examples may include antisense PNAs, antisense RNA, antisense, PMOs. This terms also encompasses RNA or DNA oligomers such as interfering RNA molecules, such as dsRNA, dsDNA, mRNA, siRNA, or hpRNA as well as locked nucleic acids, BNA, polypeptides and other oligomers and the like.

As used herein, a “FASTmer,” or “antisense FASTmer” may include an “antisense oligomer” or “antisense oligonucleotide” generated using the FAST Platform as generally described herein. In certain embodiments, a FASTmer may include any antisense molecule, such as an “antisense oligomer” that may inhibit viral gene expression or replication. Examples may include antisense PNAs, PMOs, or antisense RNAs. This term also encompasses RNA or DNA oligomers such as interfering RNA molecules, such as dsRNA, dsDNA, mRNA, siRNA, or hpRNA as well as locked nucleic acids, BNA, polypeptides and other oligomers and the like. In yet another embodiment, the PNA may include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analogs thereof.

The term “target sequence,” may mean a nucleotide sequence, such as an RNA genome sequence, that may be complementary to antisense molecules, and preferably an antisense FASTmer, such as an antisense PNA FASTmer. It will be recognized by those of skill in the art that any of the sequences herein above can be targeted by antisense inhibitors. Given the benefit of this disclosure, those of skill in the art will be able to identify a target sequence and design an antisense inhibitor oligomer, or FASTmer to target the gene or RNA sequence. Inhibition can be caused by steric interference resulting from an antisense oligomer binding the DNA or RNA sequence, thereby preventing proper transcription of the DNA sequence or activity of the RNA sequence, for example through inhibition of RNA-dependent RNA polymerase.

The term “homologous” or “sequence identity” as used herein means a nucleic acid (or fragment thereof), including morpholino nucleic acids, or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%). For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage of homology as described above is calculated as the percentage of the components found in the smaller of the two sequences that may also be found in the larger of the two sequences (with the introduction of gaps), with a component being defined as a sequence of four contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In another embodiment, the invention includes antisense PNAs that have substantial sequence similarity to another PNA. Two PNAs have “substantial sequence identity” when both of the PNAs bind to a target sequence.

As used herein, the terms “inhibit” and “inhibition” means to reduce a molecule, a reaction, an interaction, a gene, an mRNA, and/or a protein's expression, stability, function, or activity by a measurable amount, or to prevent such entirely. In one preferred embodiment, the term “inhibit” and “inhibition” means to reduce the stability, function, or activity of a RNA, and preferably inhibit the translation of a viral RNA genome, such as the ssRNA genome of SARS-CoV-2, for example, by binding to the genomic RNA sequence and sterically hindering RNA-dependent RNA polymerase progression. “Inhibitors” are compounds that, e.g., bind to, partially or totally block stimulation, decrease, prevent, delay activation, inactivate, desensitize, or down regulate a protein, a gene, and an mRNA stability, expression, function, expression, and activity, e.g., antagonists. In one preferred embodiment, the term “Inhibitors” means an antisense FASTmer that reduces the stability, function, expression, or activity of a nucleotide sequence, and preferably a RNA nucleotide sequence such as the ssRNA genome of SARS-CoV-2.

As used herein, “host” or “subject” refers to a human or animal subject. In certain preferred embodiments, the subject is a human at risk of infection by COVID-19 or infected with COVID-19. The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As used herein, the terms “complementary” or “complement” also refer to a nucleic acid comprising a sequence of consecutive nucleobases or semi-consecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%), about 98%), about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

The term “composition” or “composition of the invention” generally refers to a FASTmer, and preferably an antisense FASTmer that may be configured to be complementary to a target sequence in the ssRNA genome of the SARS-CoV-2 coronavirus. A “pharmaceutical composition” may include a nucleotide inhibitor, such as the ssRNA genome of the SARS-CoV-2 coronavirus, or FASTmer of the invention and an agent, e.g. a carrier, that may typically be used within a pharmaceutical composition for facilitating administering of the components of the pharmaceutical composition to an individual.

The term “therapeutically effective amount” as used herein refers to that amount of a FASTmer composition being administered which will relieve to some extent one or more of the symptoms of the disorder being treated. In reference to the treatment of a viral infection, and in particular a COVID-19 infection, a therapeutically effective amount refers to that amount which has the effect of (1) reducing the infection, (2) inhibiting (that is, slowing to some extent, preferably stopping) viral replication, (3) inhibiting to some extent (that is, slowing to some extent, preferably stopping) viral pathogenicity, and/or (4) relieving to some extent (or, preferably, eliminating) one or more signs or symptoms associated with the viral infection. The compositions of the invention can be used for veterinary medical purposes, as a pharmaceutical composition or as a vaccine or treatment. For example, a “therapeutically effective amount” of is a dosage of the compound that is sufficient to achieve a desired therapeutic effect. For example, a therapeutically effective amount of a compound, such as an antisense FASTmer, and preferably an antisense FASTmer targeting configured to inhibit expression of the ssRNA genome of the SARS-CoV-2 coronavirus, may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. In another embodiment, the subject receives a dosage of less than 0.1 μg/kg body weight/day, or more than 1000 mg/kg body weight/day.

In a preferred embodiment, the FASTmer of the (pharmaceutical) composition, and preferably an antisense FASTmer configured to inhibit expression of the ssRNA genome of the SARS-CoV-2 coronavirus or kit of parts according to the invention is provided in lyophilized form. Preferably, the lyophilized FASTmer is reconstituted in a suitable buffer, advantageously based on an aqueous carrier, prior to administration, e.g. Ringer-Lactate solution, which is preferred, Ringer solution, a phosphate buffer solution. In a preferred embodiment, the (pharmaceutical) composition, the FASTmer or the kit of parts according to the invention contains at least one, two, three, four, five, six or more FASTmer, preferably and preferably antisense FASTmers configured to inhibit expression of the ssRNA genome of the SARS-CoV-2 coronavirus, which are provided separately in lyophilized form (optionally together with at least one further additive) and which are preferably reconstituted separately in a suitable buffer (such as Ringer-Lactate solution) prior to their use so as to allow individual administration of each of the FASTmers.

The composition(s) of the invention may typically contain a pharmaceutically acceptable carrier. The term “pharmaceutically acceptable carrier” as used herein preferably includes the liquid or non-liquid basis of the inventive antisense FASTmer(s). If the inventive antisense FASTmer is provided in liquid form, the carrier will be water, typically pyrogen-free water; isotonic saline or buffered (aqueous) solutions, e.g phosphate, citrate etc. buffered solutions. Particularly for injection of the inventive antisense FASTmer, water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, a2C(¼, NaHCCh, a2S0₄, examples of the optional potassium salts include e.g. KCl, KI, KBr, K2CO3, KHCO3, K2SO4, and examples of calcium salts include e.g. CaCb, Cal2, CaBr2, CaCC>3, CaSC, Ca(OH)₂. Furthermore, organic anions of the aforementioned cations may be contained in the buffer.

According to a more preferred embodiment, the buffer suitable for injection purposes as defined above, may contain salts selected from sodium chloride (NaCl), calcium chloride (CaCb) and optionally potassium chloride (KCl), wherein further anions may be present additional to the chlorides. CaCb can also be replaced by another salt like KCl. Typically, the salts in the injection buffer are present in a concentration of at least 50 mM sodium chloride (NaCl), at least 3 mM potassium chloride (KCl) and at least 0.01 mM calcium chloride (CaCb). The injection buffer may be hypertonic, isotonic or hypotonic with reference to the specific reference medium, i.e. the buffer may have a higher, identical or lower salt content with reference to the specific reference medium, wherein preferably such concentrations of the afore mentioned salts may be used, which do not lead to damage of cells due to osmosis or other concentration effects. Reference media are e.g. in “in vivo” methods occurring liquids such as blood, lymph, cytosolic liquids, or other body liquids, or e.g. liquids, which may be used as reference media in “in vitro” methods, such as common buffers or liquids. Such common buffers or liquids are known to a skilled person. Ringer-Lactate solution is particularly preferred as a liquid basis.

However, one or more compatible solid or liquid fillers or diluents or encapsulating compounds may be used as well, which are suitable for administration to a person. Pharmaceutically acceptable carriers, fillers and diluents must, of course, have sufficiently high purity and sufficiently low toxicity to make them suitable for administration to a person to be treated. Some examples of compounds which can be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, com oil and oil from theobroma; polyols, such as, for example, polypropylene glycol, glycerol, sorbitol, mannitol and polyethylene glycol; alginic acid.

The choice of a pharmaceutically acceptable carrier is determined, in principle, by the manner, in which the pharmaceutical composition or antisense FASTmer according to the invention is administered. The composition or antisense FASTmer can be administered, for example, systemically or locally. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections. More preferably, composition or antisense FASTmers according to the present invention may be administered by an intradermal, subcutaneous, or intramuscular route, preferably by injection, which may be needle-free and/or needle injection. Compositions/antisense FASTmers are therefore preferably formulated in liquid or solid form. The suitable amount of the antisense FASTmers or composition according to the invention to be administered can be determined by routine experiments, e.g. by using animal models. Such models include, without implying any limitation, rabbit, sheep, mouse, rat, pig, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or antisense FASTmer is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The term “including” is used herein to mean, and is used interchangeably with, the phrase “including but not limited to.” The term “or” is used herein to mean, and is used interchangeably with, the term “and/or,” unless context clearly indicates otherwise. The terminology used herein is for describing embodiments and is not intended to be limiting. As used herein, the singular forms “a,” “and” and “the” include plural referents, unless the content and context clearly dictate otherwise. Thus, for example, a reference to “a target RNA sequence” may include a combination of two or more such target RNA sequences. Unless defined otherwise, all scientific and technical terms are to be understood as having the same meaning as commonly used in the art to which they pertain.

### TABLES

TABLE 1
Design and off-target predictions of antiviral PNA.

| | | | | Hg38 | Hg38 | Notable |
|---|---|---|---|---|---|---|
| PNA | | Target | Sequence | 1MM | 2MM | Off- |
| Name | Sequence (N to C) | Location^{#} | Conservation^{&} | OT* | OT | Targets |
| α-TRS | TAAAGTTCGTTTAGA | 65-79 | 1 mutation in 1 | 0 | 40 | |
| | (SEQ ID NO. 1) | | isolate | | | |
| α-AUG | GCTCTCCATCTTACC | 259-273 | 1 mutation in 1 | 1 | 45 | collagen type |
| | (SEQ ID NO. 2) | | isolate | | | XXV alpha 1 |
| | | | | | | chain |
| α-FS | ACACCGCAAACCCGT | 13466-13480 | No mutations | 0 | 7 | |
| | (SEQ ID NO. 3) | | | | | |
| α-PK1 | CGGGCTGCACTTACA | 13478-13492 | No mutations | 0 | 18 | |
| | (SEQ ID NO. 4) | | | | | |
| α-PK2 | TACTAGTGCCTGTGC | 13507-13521 | No mutations | 0 | 19 | |
| | (SEQ ID NO. 5) | | | | | |
| α-PK3 | GTATACGACATCAGT | 13521-13535 | No mutations | 0 | 5 | |
| | (SEQ ID NO. 6) | | | | | |

^{#}On SARS-CoV-2 genome, Genbank: MN908947.3
^{&}Among 179 clinical isolates via NCBI
*MM OT: mismatch offtarget (e.g. 0MM OT is a zeremismatch alignment to a region expected to inhibit protein expression)

### REFERENCES

1. WHO Coronavirus Disease (COVID-19) Dashboard (Nov. 9, 2020).
2. Antiviral Drugs That Are Approved or Under Evaluation for the Treatment of COVID-19 (Nov. 9, 2020).
3. K. E. Jones, et al., Global trends in emerging infectious diseases. Nature 451, 990-993 (2008).
4. E. S. Pronker, T. C. Weenen, H. Commandeur, E. H. J. H. M. Claassen, A. D. M. E. Osterhaus, Risk in Vaccine Research and Development Quantified. PLoS One 8, e57755 (2013).
5. J. P. Martinez, F. Sasse, M. Brönstrup, J. Diez, A. Meyerhans, Antiviral drug discovery: broad-spectrum drugs from nature. Nat. Prod. Rep. 32, 29-48 (2015).
6. K. L. Warfield, et al., Gene-specific countermeasures against Ebola virus based on antisense phosphorodiamidate morpholino oligomers. PLoS Pathog. 2, el (2006).
7. Y. Wu, et al., Inhibition of highly pathogenic avian H5N1 influenza virus replication by RNA oligonucleotides targeting NS1 gene. Biochem. Biophys. Res. Commun. 365, 369-374 (2008).
8. B. Li, et al., Using siRNA in prophylactic and therapeutic regimens against SARS coronavirus in Rhesus macaque. Nat. Med. 11, 944-951 (2005).
9. Z. Zeng, et al., A Tat-conjugated Peptide Nucleic Acid Tat-PNA-DR Inhibits Hepatitis B Virus Replication In Vitro and In Vivo by Targeting LTR Direct Repeats of HBV RNA. Mol. Ther.—Nucleic Acids 5, e295 (2016).
10. R. Burrer, et al., Antiviral Effects of Antisense Morpholino Oligomers in Murine Coronavirus Infection Models. J. Virol. 81, 5637 LP-5648 (2007).
11. D.-G. Ahn, et al., Interference of ribosomal frameshifting by antisense peptide nucleic acids suppresses SARS coronavirus replication. Antiviral Res. 91, 1-10 (2011).
12. B. W. Neuman, et al., Inhibition, escape, and attenuated growth of severe acute respiratory syndrome coronavirus treated with antisense morpholino oligomers. J. Virol. 79, 9665-9676 (2005).
13. B. D. Gildea, J. M. Coull, Methods for Modulating the Solubility of Synthetic Polymers (2004).
14. E. L. Hatcher, et al., Virus Variation Resource—improved response to emergent viral outbreaks. Nucleic Acids Res. 45, D482-D490 (2017).
15. K. Cleal, L. He, P. D. Watson, a T. Jones, Endocytosis, intracellular traffic and fate of cell penetrating peptide based conjugates and nanoparticles. Curr Pharm Des 19, 2878-2894 (2013).
16. R. L. Juliano, X. Ming, K. Carver, B. Laing, Cellular Uptake and Intracellular Trafficking of Oligonucleotides: Implications for Oligonucleotide Pharmacology. 24 (2014).

SEQUENCE LISTING
SEQ ID NO. 1
Peptide Nucleic Acid
α-TRS
Artificial
TAAAGTTCGTTTAGA
SEQ ID NO. 2
Peptide Nucleic Acid
α-AUG
Artificial
GCTCTCCATCTTACC
SEQ ID NO. 3
Peptide Nucleic Acid
α-FS
Artificial
ACACCGCAAACCCGT
SEQ ID NO. 4
Peptide Nucleic Acid
α-PK1
Artificial
CGGGCTGCACTTACA
SEQ ID NO. 5
Peptide Nucleic Acid
α-PK2
Artificial
TACTAGTGCCTGTGC
SEQ ID NO. 6
Peptide Nucleic Acid
α-PK3
Artificial
GTATACGACATCAGT
SEQ ID NO. 7
RNA
TRS target sequence
SAPS-CoV-2
UCUAAACGAACUUUA
SEQ ID NO. 8
RNA
AUG target sequence
SAPS-CoV-2
GGUAAGAUGGAGAGC
SEQ ID NO. 9
RNA
FS target sequence
SAPS-CoV-2
CGGGUUUGCGGUGU
SEQ ID NO. 10
RNA
PK-1 target sequence
SAPS-CoV-2
UGUAAGUGCAGCCCG
SEQ ID NO. 11
RNA
PK-2 target sequence
SAPS-CoV-2
AUGAUCACGGACACG
SEQ ID NO. 12
RNA
PK-3 target sequence
SAPS-CoV-2
CAUAUGCUGUAGUCA

1. A method of treating or preventing a SARS-CoV-2 (COVID-19) coronavirus infection in a subject in need thereof, comprising the step of administering a therapeutically effective amount of at least one antisense FASTmer complementary to a genomic target sequence, motif or structure of SARS-CoV-2, and wherein said antisense oligomer binds to, and inhibits SARS-CoV-2 viral genome expression.

2. (canceled)

3. The method of claim 2, wherein said subject is a human infected with COVID-19, or at risk of being infected with COVID-19.

4. The method of claim 1, wherein said genomic target sequence, motif or structure comprises a genomic target sequence, motif or structure selected from the group consisting of: the transcriptional regulatory sequence (TRS), the start codon region (AUG), the polyprotein 1AB frameshift motif (FS), and the pseudoknot structure (PKI).

5. The method of claim 1, wherein said genomic target sequence comprises a genomic target sequence selected from the group consisting of: the nucleotide sequence according to SEQ ID NOs. 7-12.

6. The method of claim 1, wherein said target sequence comprises a target sequence involved in viral transcription or translation.

7. The method of claim 1, wherein said target sequence comprises a target sequence that is conserved across two or more species of coronavirus.

8. The method of claim 1, wherein said antisense FASTmer comprises an antisense peptide nucleic acid (PNA) FASTmer.

9. The method of claim 8, wherein said antisense PNA FASTmer comprises an antisense PNA FASTmer selected from the group consisting of: the nucleotide sequence according to SEQ ID NOs. 1-6.

10. The method of claim 8, wherein said antisense PNA FASTmer comprises an antisense PNA FASTmer configured to be complementary to a target sequence of SARS-CoV-2 having a nucleotide sequence selected from the group consisting of: α-TRS (SEQ ID NO. 1), α-AUG (SEQ ID NO. 2), α-FS (SEQ ID NO. 3), and α-PK1 (SEQ ID NO. 4), α-PK2 (SEQ ID NO. 5), and α-PK13 (SEQ ID NO. 6).

11. A pharmaceutical composition comprising at least one antisense FASTmer complementary to a target sequence, motif, or structure of SARS-CoV-2, and wherein said antisense FASTmer inhibits SARS-CoV-2 viral genome expression, and a pharmaceutically acceptable carrier.

12. The composition of claim 11, wherein said genomic target sequence, motif or structure comprises a genomic target sequence, motif or structure selected from the group consisting of: the transcriptional regulatory sequence (TRS), the start codon region (AUG), the polyprotein 1AB frameshift motif (FS), and the pseudoknot structure (PKI).

13. The composition of claim 11, wherein said genomic target sequence comprises a genomic target sequence selected from the group consisting of: the nucleotide sequence according to SEQ ID NOs. 7-12.

14. The composition of claim 11, wherein said target sequence comprises a target sequence involved in viral transcription or translation.

15. The composition of claim 11, wherein said antisense FASTmer comprises an antisense peptide nucleic acid (PNA) FASTmer.

16. The composition of claim 15 wherein said antisense PNA FASTmer comprises an antisense PNA FASTmer selected from the group consisting of: the nucleotide sequence according to SEQ ID NOs. 1-6.

17. The composition of claim 15, wherein said antisense PNA FASTmer comprises an antisense PNA FASTmer configured to be complementary to a target sequence of SARS-CoV-2 having a nucleotide sequence selected from the group consisting of: α-TRS (SEQ ID NO. 1), α-AUG (SEQ ID NO. 2), α-FS (SEQ ID NO. 3), and α-PK1 (SEQ ID NO. 4), α-PK2 (SEQ ID NO. 5), and α-PK13 (SEQ ID NO. 6).

18. A method of treating or preventing COVID-19 coronavirus infection in a subject in need thereof, comprising the step of administering a therapeutically effective amount of the composition of claim 17.

19-25. (canceled)

26. An isolated PNA oligomer selected from the group consisting of: SEQ ID NOs. 1-6.

27. A pharmaceutical composition comprising at least one PNA oligomer of claim 26, and a pharmaceutically acceptable carrier.

28. A method of treating or preventing COVID-19 coronavirus infection in a subject in need thereof, comprising the step of administering a therapeutically effective amount of the pharmaceutical composition of claim 27.


SEQUENCE LISTING (12 sequences)

SEQ ID NO: 1 - DNA, 15 bp, Artificial
  alpha-TRS Peptide Nucleic Acid
taaagttcgtttaga

SEQ ID NO: 2 - DNA, 15 bp, Artificial
  alpha-AUG Peptide Nucleic Acid
gctctccatcttacc

SEQ ID NO: 3 - DNA, 15 bp, Artificial
  alpha-FS Peptide Nucleic Acid
acaccgcaaacccgt

SEQ ID NO: 4 - DNA, 15 bp, Artificial
  alpha-PK1 Peptide Nucleic Acid
cgggctgcacttaca

SEQ ID NO: 5 - DNA, 15 bp, Artificial
  alpha-PK2 Peptide Nucleic Acid
tactagtgcctgtgc

SEQ ID NO: 6 - DNA, 15 bp, Artificial
  alpha-PK3 Peptide Nucleic Acid
gtatacgacatcagt

SEQ ID NO: 7 - RNA, 15 nt, SARS-CoV-2
ucuaaacgaacuuua

SEQ ID NO: 8 - RNA, 15 nt, SARS-CoV-2
gguaagauggagagc

SEQ ID NO: 9 - RNA, 14 nt, SARS-CoV-2
cggguuugcggugu

SEQ ID NO: 10 - RNA, 15 nt, SARS-CoV-2
uguaagugcagcccg

SEQ ID NO: 11 - RNA, 15 nt, SARS-CoV-2
augaucacggacacg

SEQ ID NO: 12 - RNA, 15 nt, SARS-CoV-2
cauaugcuguaguca